(19) United States
(12) Patent Application Publication
(10) Pub. No.: US 2025/0154096 A1
(43) Pub. Date: May 15, 2025

(54) SPHINGOLIPID PRODUCTION

(71) Applicant: CATAYA BIO (SHANGHAI) CO., LTD., Shanghai (CN)

(72) Inventors: Gyula DEKANI, COIMBRA (PT); Györgyi OSZTROVSZKY, COIMBRA (PT); Ferenc HORVATH, COIMBRA (PT)

(73) Assignee: CATAYA BIO (SHANGHAI) CO., LTD., Shanghai (CN)

(21) Appl. No.: 18/840,015

(22) PCT Filed: Feb. 17, 2023
(86) PCT No.: PCT/EP2023/054051

§ 371 Date: Aug. 20, 2024

(30) Foreign Application Priority Data
Feb. 21, 2022 (PT) 117803

(51) Int. Cl. C07C 233/20

(52) U.S. Cl. CPC C07C 233/20

(57) ABSTRACT

The present invention relates to a novel and efficient method for the production of sphingolipids via the N-acylation of lysosphingolipids such as D-eritro-sphingosine or 6-hydroxy D-eritro-sphingosine, analogues thereof, or salts thereof. The method comprises the use of esters as acylating agents and is especially suitable for the production of ceramides.

### CROSS REFERENCE TO RELATED APPLICATIONS

This is the U.S. National Stage of International Application No. PCT/EP2023/054051, filed Feb. 17, 2023, which was published in English under PCT Article 21(2), which in turn claims the benefit of Portuguese Application No. 117803, filed Feb. 21, 2022.

### FILED OF THE INVENTION

The present invention relates to a novel and efficient method for the production of sphingolipids via the N-acylation of lysosphingolipids such as D-eritro-sphingosine or 6-hydroxy D-eritro-sphingosine, analogues thereof, or salts thereof. The method is based on the use of esters as acylating agents and is especially suitable for the production of ceramides and glycosphingolipids.

### BACKGROUND

Sphingolipids are an important class of polar lipids mainly found on the surface of eukaryotic cells. Sphingolipids are structurally characterized by a sphingoid base backbone and can be divided in different classes such as, ceramides, and glycosphingolipids.

Ceramides are N-acylated sphingoid bases lacking additional head groups at the 1-position of the sphingoid base backbone, and wherein the N-acyl group of ceramides typically derives from a fatty acid. Glycosphingolipids (GSLs) are glycoconjugates deriving from ceramides, wherein a glycan moiety is linked to the 1-hydroxyl group of a ceramide via a glycosidic linkage.

Sphingolipids are involved in diverse biological processes and play important structural and functional roles such as cell-cell recognition, communication, and intercellular adhesion. Particularly, GSLs such as gangliosides are found in the brain and play roles in neurological diseases, whereas ceramides are the main constituent of the stratum corneum lipid layer and have a major role in the water-retaining properties of the epidermis, as well as in the barrier function of the skin.

Accordingly, sphingolipids hold great potential as therapeutics, cosmetics, and as tools for the study of important biological processes. However, they are not readily available for fundamental and clinical research. In fact, sphingolipids such as ceramides and GSLs are characterized by a high structural complexity and their preparation represents a challenge.

Sphingolipids such as ceramides and GSLs may be obtained via the N-acylation of lysosphingolipids.

Typically, lysosphingolipids are defined as sphingolipid breakdown products which lack the amide-linked fatty acid at the 2-position of the sphingoid base backbone. Accordingly, for each parental sphingolipid there is a corresponding lysosphingolipid that has an identical head group at the 1-position but lacks the amide-bound fatty acid at the 2-position (Hannun et al., Science 1989, 243, 500-507).

N-acylation of lysosphingolipids may be performed via chemical or enzymatic approaches.

Current chemical N-acylation methods are typically based on the use of amide bond coupling reagents such as N-hydroxysuccinimide (NHS), 1,3-dicyclohexylcarbodiimide (DCC), or 1-ethyl-3-(3-dimethylaminopropyl) carbodiimide (EDC) (Skolova et al, Biochim Biophys Acta Biomembr. 2017, 1859, 824-834, WO0172701 A1), or sulfonyl chloride (U.S. Pat. No. 5,631,356 A). Alternatively, esters of acids have been utilized for the N-acylation of phytosphingosine and dihydrosphingosine (WO2013038985 A1, WO2013094108 A1, EP0856510 A1, EP0835864A2, Drug. Del. Sci. Tech. 2014, 24, 689-693, Org. Process Res. Dev. 2019, 23, 452-461).

Current chemical approaches for the N-acylation of lysosphingolipids possess several disadvantages, these include the use of expensive and/or toxic coupling reagents which require the use of water-free solvents and/or performing the reaction under exclusion of air.

Current enzymatic approaches are based on the use of lipases (WO1994026919A1, US2011077302A1) which, however, are typically not specific for the amino group of a lysosphingolipid but may also act on hydroxyl groups, thus leading to the formation of mixtures of N- and O-acylation products, and thus requiring a lengthily and costly purification of the target compound.

### SUMMARY OF THE INVENTION

In a first aspect, the present invention relates to a method for the production of a sphingolipid of formula (1):

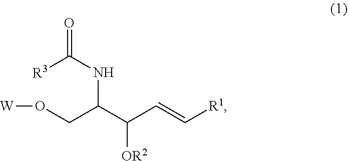

wherein
W is H or a glycosyl moiety,
R¹ is H, aryl, or a C₁₋₅₀ alkyl, preferably a C₁₋₁₅ alkyl, more preferably a C₁₀₋₁₅ alkyl, which may be saturated or contain one or more double and/or triple bonds, and/or which may contain one or more functional groups, the functional group being preferably selected from the group consisting of a hydroxyl group, an alkoxy group, an acyloxy group, an acylamido group, a thiol, a thioether or a phosphorus-containing functional group,
R² is H, a substituted or unsubstituted C₁₋₆ alkyl, or a substituted or unsubstituted C₁₋₆ acyl,
R³ is selected from a substituted or unsubstituted aryl, a heteroalkyl, a substituted or unsubstituted C₁₋₃₁ alkyl which may be saturated or unsaturated, preferably a substituted or unsubstituted C₉₋₃₁ alkyl which may be saturated or unsaturated;
from a lysosphingolipid of formula (2), or a salt thereof:

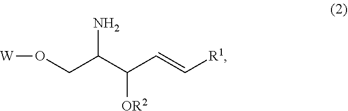

wherein:
W, R¹, and R² are as defined as for the sphingolipid of formula (1), and
the method comprising reacting the lysosphingolipid salt of formula (2), or the salt thereof with an ester of formula (3):

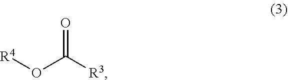

wherein
R³ is as defined as for the sphingolipid of formula (1), and
R⁴ is a C₁-C₄ alkyl, preferably selected from methyl, ethyl, propyl, isopropyl, butyl, or isobutyl, more preferably selected from methyl, or ethyl:
in the presence of a base, in an alcohol solvent.

### DETAILED DESCRIPTION OF INVENTION

The present inventors have established for the first time an efficient and economic method for the chemical N-acylation of lysosphingolipids such as D-eritro-sphingosine or 6-hydroxy D-eritro-sphingosine, analogues thereof, or salts thereof, and wherein the lysosphingolipid used as the starting material is preferably obtained via synthetic and/or biotechnological approaches. Surprisingly, the present inventors have found that lysosphingolipids such as D-eritro-sphingosine or 6-hydroxy D-eritro-sphingosine, analogues thereof, or salts thereof can be N-acylated using esters of formula (3):

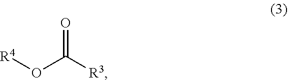

wherein
R³ is as defined as for the sphingolipid of formula (1), and
R⁴ is a C₁-C₄ alkyl, preferably selected from methyl, ethyl, propyl, isopropyl, butyl, or isobutyl, more preferably selected from methyl, or ethyl:
in the presence of a base, in an alcohol solvent.

Non-limiting embodiments of different aspects of the invention are described below and illustrated by non-limiting examples.

The terms, definitions and embodiments described throughout specification of the invention relate to all aspects and embodiments of the invention.

The term “a” grammatically is a singular, but it may as well mean the plural of e.g., the intended compound. For example, a skilled person would understand that in the expression “a lysosphingolipid of formula (2)”, the provision of not only one single a lysosphingolipid of formula (2), but of a variety of lysosphingolipids of the same type is meant.

In formulas representing a moiety, or a group such as for example the alkyl of formula (5) or (6) the symbol [symbol] means a point of attachment to another group or atom.

As used herein, the various functional groups or substituents represented will be understood to have a point of attachment at the functional group or atom having the dash (—). For example, in the case of —OH it will be understood that the point of attachment is the oxygen atom. If a group is listed without a dash, then the attachment point is indicated by the plain and ordinary meaning of the recited group.

The letters H, Cl, Br, I and F refer to a hydrogen, a chlorine, a bromine, an iodine, and a fluorine atom respectively.

The skilled person would understand that when speaking of position C-1, C-2, C-3, C-4, C-5 etc., reference is herein always made to the respective carbon atoms of sphingolipids of formula (1), or lysosphingolipids of formula (2), (7), or (8). Positions C-1, C-2, C-3, C-4, C-5 may also be referred to as 1-position, 2-position, 3-position, 4-position, and 5-position respectively.

As used herein, the term “alkyl” refers to an acyclic straight or branched hydrocarbyl group having 1-50 carbon atoms which may be saturated or contain one or more double and/or triple bonds (so, forming for example an alkenyl or an alkynyl), and/or which may be substituted or unsubstituted, as herein further described. Examples of “alkyl” include, but are not limited to, methyl, ethyl, n-propyl, isopropyl, isobutyl, n-butyl, sec-butyl, tert-butyl, isopentyl, n-pentyl, neo-pentyl, n-hexyl, ethenyl, propenyl, 1-butenyl, 2-butenyl, isobutenyl, 1-pentenyl, 2-pentenyl, 2-methyl-1-butenyl, 3-methyl-1-butenyl, 2-methyl-2-butenyl, 1-hexenyl, 2-hexenyl, 3-hexenyl, methylpentenyl, dimethylbutenyl, ethynyl, propynyl, 1-butynyl, 2-butynyl, pentynyl, and hexynyl, each of which may be substituted or unsubstituted. Typically, the term alkyl refers to a straight acyclic hydrocarbyl group having 1-31 carbons, which may be substituted or unsubstituted.

As used herein, the term “aryl” refers to an aromatic cyclic hydrocarbyl group having 5-14 ring carbon atoms, which may be mono- or polycyclic, which may contain fused rings, preferably 1 to 3 fused or unfused rings, and which may contain one or more heteroatoms, and/or which may be substituted or unsubstituted, as herein further described. Examples of “aryl” include, but are not limited to, phenyl, naphtyl, anthracyl, phenantryl, pyrrolyl, imidazolyl, thiophenyl, furanyl, oxazolyl, thiazolyl, pyridinyl, pyrimidinyl, pyrazinyl, triazinyl, and benzofuranyl, each of which may be substitute or unsubstituted. Typically, the term “aryl” refers to a substituted or unsubstituted phenyl.

As used herein, the term “acyl” refers to a group derived by the removal of one or more hydroxyl group from an oxoacid, preferably from a carboxylic acid. The acyl group according to the present invention is typically a saturated or unsaturated C₂₋₃₂ acyl, which may be substitute or unsubstituted.

As used herein the term “alkylene” refers to a bivalent saturated or unsaturated aliphatic radical deriving from a substituted or unsubstituted alkane by removal of two hydrogen atoms from different carbon atoms, preferably from the terminal carbon atoms. The alkylene according to the present invention, typically is a C₆₋₂₈ alkylene which may be saturated or contain one or more double and/or triple bonds, and/or which may be substituted or unsubstituted, as herein further described.

As used herein, the term “substituted” means that the group in question is substituted with a group which typically modifies the general chemical characteristics of the group in question. The substituents can be used to modify characteristics of the molecule, such as molecule stability, molecule solubility and the ability of the molecule to form crystals. The person skilled in the art will be aware of other suitable substituents of a similar size and charge characteristics, which could be used as alternatives in a given situation.

In connection with the terms “alkyl”, “aryl”, “acyl”, and “alkylene” the term substituted means that the group in question is substituted one or several times, preferably 1 to 3 times, with group(s) selected from hydroxy (which when bound to an unsaturated carbon atom may be present in the tautomeric keto form), oxo, C₁₋₆-alkoxy (i.e. C₁₋₆-alkyl-oxy), C₂₋₆-alkenyloxy, carboxy, oxo, C₁₋₆-alkoxycarbonyl, C₁₋₆-alkylcarbonyl, formyl, aryl, aryloxycarbonyl, aryloxy, arylamino, arylcarbonyl, heteroaryl, heteroarylamino, heteroaryloxycarbonyl, heteroaryloxy, heteroarylcarbonyl, amino, mono- and di(C₁₋₆-alkyl)amino, carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, cyano, guanidino, carbamido, C₁₋₆-alkyl-sulphonyl-amino, aryl-sulphonyl-amino, heteroaryl-sulphonyl-amino, C₁₋₆-alkanoyloxy, C₁₋₆-alkyl-sulphonyl, C₁₋₆-alkyl-sulphinyl, C₁₋₆-alkylsulphonyloxy, nitro, C₁₋₆-alkylthio, halogen, where any alkyl, alkoxy, and the like representing substituents may be substituted with hydroxy, C₁₋₆-alkoxy, C₂₋₆-alkenyloxy, carboxy, C₁₋₆-alkylcarbonylamino, halogen, C₁₋₆-alkylthio, C₁₋₆-alkyl-sulphonyl-amino, or guanidino.

The term “lysosphingolipid” when used herein refers to a sphingolipid which lacks the amide-linked fatty acid at the C-2 position of the sphingoid base backbone. Suitable lysosphingolipids, for use in the context of the present invention are sphingoid bases, glycosylated sphingoid bases, and analogs thereof and may be represented by a lysosphingolipid of formula (2):

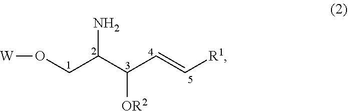

wherein
W is H or a glycosyl moiety,
R¹ is H, aryl, or a C₁₋₅₀ alkyl, preferably a C₁₋₁₅ alkyl, more preferably a C₁₀₋₁₅ alkyl, which may be saturated or contain one or more double and/or triple bonds, and/or which may contain one or more functional groups, the functional group being preferably selected from the group consisting of a hydroxyl group, an alkoxy group, an acyloxy group, an acylamido group, a thiol, a thioether or a phosphorus-containing functional group,
R² is H, a substituted or unsubstituted C₁₋₆ alkyl, or a substituted or unsubstituted C₁₋₆ acyl.

The term “glycosyl moiety” when used herein is defined to encompass a moiety derived from a monosaccharide or from an oligosaccharide (more than one monosaccharide units), wherein the anomeric carbon of the monosaccharide or the anomeric carbon at the reducing end of the oligosaccharide is engaged in a glycosidic bond with another chemical entity, and the bond, if not further specified, may be an alpha or a beta glycosidic bond. A glycosyl moiety having more than one monosaccharide unit may represent a linear or a branched structure. The monosaccharide unit can be any 5-9 carbon atom sugar, comprising aldoses (e.g. D-glucose, D-galactose, D-mannose, D-ribose, D-arabinose, L-arabinose, D-xylose, etc.), ketoses (e.g. D-fructose, D-sorbose, D-tagatose, etc.), deoxysugars (e.g. L-rhamnose, L-fucose, etc.), deoxy-aminosugars (e.g. N-acetylglucosamine, N-acetylmannosamine, N-acetylgalactosamine, etc.), uronic acids, ketoaldonic acids (e.g. sialic acid). The monosaccharide unit can form different cyclic structures such as pyranose (six-membered) cyclic structures or furanose (five-membered) cyclic structures.

The glycosyl moieties according to the present invention may be illustrated in the following style:

Galβ1-4Glc1-, wherein the dash (—) represents the point of attachment of the glycosyl moiety and wherein the glycosyl moiety may be linked via an alpha or a beta glycosidic bond, preferably a beta glycosidic bond.

In the context of the present invention, the terms “about”, “around”, or “approximate” are applied interchangeably to a particular value (e.g. “a temperature of about 25° C.”, “a temperature of around 25° C.”, or “a temperature of approximate 25° C.”), or to a range (e.g. “an amount from about 0.1 to about 0.2”, “an amount from around 0.1 to around 0.2”, or “an amount from approximate 0.1 to approximate 0.2”), to indicate a deviation from 0.1% to 10% of that particular value.

Suitable esters for use in the context of the present invention typically derive from a carboxylic acid and are represented by formula (3).

In some embodiments, R³ of the ester of formula (3) is a substituted or unsubstituted C₉₋₃₁ alkyl of formula (5), or (6):

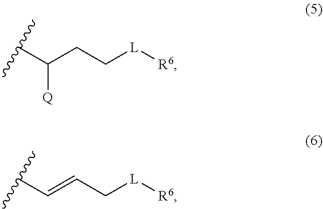

wherein
Q is selected from H, —OH, Cl, Br, I, or F,
L is a linear C₆₋₂₈ alkylene, which may be saturated or contain one or more double and or triple bonds,
R⁶ is H, or —OR⁷, wherein R⁷ is hydrogen or a linear C₂₋₃₀ acyl which may be saturated or contain one or more double bonds.

In some preferred embodiments, R³ of the ester of formula (3) is a substituted or unsubstituted C₉₋₃₁ alkyl of formula (5).

Accordingly, in some preferred embodiments, the ester of formula (3) is an ester of formula (9):

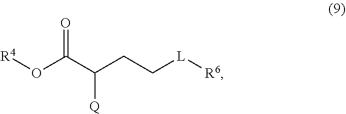

wherein
R⁴ is a C₁-C₄ alkyl, preferably selected from methyl, ethyl, propyl, isopropyl, butyl, or isobutyl, more preferably selected from methyl, or ethyl;
Q is selected from H, —OH, Cl, Br, I, or F, preferably selected from H, or —OH.
L is a linear C₆₋₂₈ alkylene, which may be saturated or contain one or more double and or triple bonds,
R⁶ is H, or —OR⁷, wherein R⁷ is hydrogen or a linear C₂₋₃₀ acyl which may be saturated or contain one or more double bonds.

In some embodiments, for the alkyl group of formula (5) and for the ester of formula (9) Q is H, L is a straight-chain saturated unsubstituted C₁₄-C₂₈ alkylene, and R⁶ is H.

In some embodiments the ester of formula (3), or the ester of formula (9) is an ester deriving from a C₁₈-C₂₆ non-hydroxy fatty [N].

In some embodiments, the ester of formula (3) or the ester of formula (9) is an ester deriving from stearic acid [N (18:0)].

In some embodiments, the ester of formula (3) or the ester of formula (9) is an ester deriving from eicosanoic acid [N (20:0)].

In some embodiments, the ester of formula (3) or the ester of formula (9) is an ester deriving from behenic acid [N (22:0)].

In some embodiments, the ester of formula (3) or the ester of formula (9) is an ester deriving from lignoceric acid [N (24:0)].

In some embodiments, the ester of formula (3) or the ester of formula (9) is an ester deriving from hexacosanoic acid [N (26:0)].

In some embodiments, for the alkyl group of formula (5) and for the ester of formula (9) Q is —OH, L is a straight-chain saturated unsubstituted C₁₄-C₂₈ alkylene, and R⁶ is H.

In some embodiments the ester of formula (3), or the ester of formula (9) is an ester deriving from a C₈-C₂₆ α-hydroxy fatty acid [A].

In some embodiments, the ester of formula (3) or the ester of formula (9) is an ester deriving from α-hydroxystearic acid [A (18:0)].

In some embodiments, the ester of formula (3) or the ester of formula (9) is an ester deriving from α-hydroxyeicosanoic acid [A (20:0)].

In some embodiments, the ester of formula (3) or the ester of formula (9) is an ester deriving from α-hydroxybehenic acid [A (22:0)].

In some embodiments, the ester of formula (3) or the ester of formula (9) is an ester deriving from α-hydroxylignoceric acid [A (24:0)].

In some embodiments, the ester of formula (3) or the ester of formula (9) is an ester deriving from α-hydroxyhexacosanoic acid [A (26:0)].

In some embodiments, for the alkyl group of formula (5) and for the ester of formula (9) Q is H, L is a straight-chain saturated unsubstituted C₁₄-C₂₈ alkylene, and R⁶ is —OR⁷ wherein R⁷ is a saturated or unsaturated C₁₈ acyl.

The letters in brackets refer to the shorthand nomenclature developed by Motta et al., Biochim Biophys Acta., 1993, 1182:147-151 and expanded by Rabionet et al., Biochim Biophys Acta, 2014, 1841:422-434, and by Masukawa et al., Journal of Lipid Research, 2008, 49, 1466-1476, wherein non-hydroxy fatty acids (N), alpha-hydroxy fatty acids (A), and omega-linoleoyloxy fatty acids (EO), may be represented by the letters N, A, and EO, respectively, and wherein the number of carbons and unsaturations may be expressed in parentheses following the letters of N, A, E, and O.

In some embodiments, R⁴ of the ester of formula (3) or (9) is a methyl.

In some embodiments, the ester of formula (3) or (9) is a methyl ester selected from the group consisting of stearic acid methyl ester, eicosanoic acid methyl ester, behenic acid methyl ester, lignoceric acid methyl ester, and hexacosanoic acid methyl ester.

In some embodiments, the ester of formula (3) or (9) is a methyl ester selected from the group consisting of α-hydroxystearic acid methyl ester, α-hydroxyeicosanoic acid methyl ester, α-hydroxybehenic acid methyl ester, α-hydroxylignoceric acid methyl ester, α-hydroxyhexacosanoic acid methyl ester.

In some embodiments, R⁴ of the ester of formula (3) is a substituted or unsubstituted C₉₋₃₁ alkyl of formula (6).

Accordingly, in some embodiments, the ester of formula (3) is an ester of formula (10):

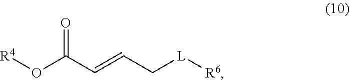

wherein
R⁴ is a C₁-C₄ alkyl, preferably selected from methyl, ethyl, propyl, isopropyl, butyl, or isobutyl, more preferably selected from methyl, or ethyl;
L is a linear C₆₋₂₈ alkylene, which may be saturated or contain one or more double and or triple bonds,
R⁶ is H, or —OR⁷, wherein R⁷ is hydrogen or a linear C₂₋₃₀ acyl which may be saturated or contain one or more double bonds.

Esters for use in the context of the present invention may be purchased from established manufacturers (e.g. Merck), or may be synthesized according to methods known to the skilled person such as that described by Wretensjo et al., J. Chromatogr. 1990, 152, 89-97.

The present invention describes a method for the production of a sphingolipid of formula (1), wherein an ester of formula (3), (9), or (10) is reacted with a lysosphingolipid of formula (2), (7), or (8) in the presence of a base.

Lysosphingolipids for use in the context of the present invention are preferably obtained via synthetic and/or biotechnological approaches such as those described in WO 2021170624 A2; or in WO2019238970 A1; or by Wisse et al., J. Org. Chem. 2015, 80 (14), 7258-7265; or by Yadav et al., Tetrahedron Lett. 2003, 44, 2983-2985.

In some embodiments, the lysosphingolipid of formula (2) is a lysosphingolipid of formula (7), or a salt thereof:

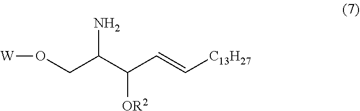

wherein
W is H or a glycosyl moiety,
R² is H, a substituted or unsubstituted C₁₋₆ alkyl, or a substituted or unsubstituted C₁₋₆ acyl,
In some embodiments, the stereochemical configuration of the C-2, C-3, and C-4 carbon atoms of the lysosphingolipid of formula (7) is (2S,3R,4E).

In some preferred embodiments, the lysosphingolipid of formula (7) is D-erythro-sphingosine, or a salt thereof.

In some embodiments, the lysosphingolipid of formula (2) is a lysosphingolipid of formula (8), or a salt thereof:

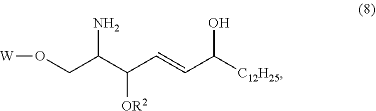

wherein
W is H or a glycosyl moiety,
R² is H, a substituted or unsubstituted C₁₋₆ alkyl, or a substituted or unsubstituted C₁₋₆ acyl.

In some embodiments the stereochemical configuration of the C-2, C-3, and C-4 carbon atoms of the lysosphingolipid of formula (8) is (2S,3S,4E).

In some preferred embodiments, the lysosphingolipid of formula (8) is 6-hydroxy-D-ribo-sphingosine.

In some embodiments, lysosphingolipids of formula (2), (7), or (8) may be produced or utilized in the form of salts, preferably in the form of pharmaceutical acceptable salts.

In some embodiments, the salts of lysosphingolipids of formula (2), (7), or (8), may be formed from the following acids: hydrochloric acid, hydrobromic acid, sulfuric acid, phosphoric acid, polyphosphoric acid, acetic acid, camphor sulfonic acid, p-toluene sulfonic acid, methane sulfonic acid, trifluoromethanesulfonic acid, perchloric acid.

Typically, the lysosphingolipid of formula (2), (7), or (8) and the ester of formula (3), (9) or (10) are reacted in the presence of a base such as an alkoxide.

Alkoxides for use in the contest of the present invention may be represented by an alkoxide of formula (4):

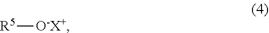

wherein
R⁵ is a C₁-C₄ alkyl, preferably selected from methyl, ethyl, propyl, isopropyl, butyl, or isobutyl, more preferably selected from methyl, or ethyl;
X⁺ is a cation selected from Na⁺, K⁺, Li⁺ or NH₄⁺, preferably Na⁺.

In some more preferred embodiments, the lysosphingolipid of formula (2), (7), or (8) and the ester of formula (3), (9) or (10) are reacted in the presence of sodium methoxide.

The base may be used in catalytic amounts, equimolar amounts or in excess.

In some embodiments, the lysosphingolipid of formula (2), (7), or (8) is in the free-base form and the base is used in a catalytic amount from about 0.1 to about 0.5 molar equivalents based on the amount of the lysosphingolipid, preferably the base is used in a catalytic amount from about 0.1 to about 0.2 molar equivalents based on the amount of the lysosphingolipid.

In some embodiments, the lysosphingolipid of formula (2), (7), or (8) is in a salt form and the base is used in an amount from about 0.5 to about 1.7 molar equivalents based on the amount of the lysosphingolipid, preferably the base is used in an amount from about 1.2 to 1.4 molar equivalents based on the amount of the lysosphingolipid.

Typically, the lysosphingolipid, the ester, and the base are reacted in an alcohol solvent such as methanol, ethanol, propanol, isopropanol, butanol, or isobutanol.

In some preferred embodiments, the reaction is performed in methanol. In some embodiments the reaction is performed in a mixture of one or more alcohol solvents, such as a mixture of methanol and ethanol, methanol and propanol, methanol and isopropanol, methanol and butanol, methanol and isobutanol, or the mixture of water and an aliphatic alcohol.

The lysosphingolipid, the ester, and the base are typically reacted at a temperature from about 50° C. to about 125° C. Preferably at a temperature from about 60° C. to about 65° C.

Accordingly, in some preferred embodiments, the reaction is performed at a temperature of about 60° C., 61° C., 62° C., 63° C., 64° C., or 65° C.

The components of the reactions of the invention may be combined in any order, and it will be appreciated that the order of combining the reactants may be adjusted as needed.

For example, the base may be added to a solution of the lysosphingolipid and the ester. As another example, a solvent may be added to a flask containing the lysosphingolipid and the ester, followed by the base.

The lysosphingolipid, the ester, and the base, as well as any other reagent used during the reaction may be added to the reaction either as a solid or dissolved in a solvent, and in any quantities and manner effective for the intended result of the reaction.

The reaction between the ester, the base and the lysosphingolipid according to the present invention results in the selective N-acylation of the amino group at the C-2 carbon atom of the lysosphingolipid of formula (2), (7), or (8), thereby producing a sphingolipid of formula (1).

In some embodiments, the sphingolipid of formula (1) is a sphingolipid of formula (11):

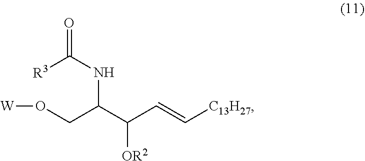

wherein
W is H or a glycosyl moiety,
R² is H, a substituted or unsubstituted C₁₋₆ alkyl, or a substituted or unsubstituted C₁₋₆ acyl,
R³ is selected from a substituted or unsubstituted aryl, a heteroalkyl, a substituted or unsubstituted C₁₋₃₁ alkyl which may be saturated or unsaturated, preferably a substituted or unsubstituted C₉₋₃₁ alkyl which may be saturated or unsaturated.

In some embodiments, the sphingolipid of formula (1) is a sphingolipid of formula (12):

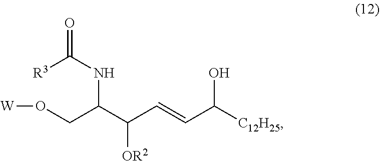

wherein
W is H or a glycosyl moiety, R² is H, a substituted or unsubstituted C₁₋₆ alkyl, or a substituted or unsubstituted C₁₋₆ acyl,
R³ is selected from a substituted or unsubstituted aryl, a heteroalkyl, a substituted or unsubstituted C₁₋₃₁ alkyl which may be saturated or unsaturated, preferably a substituted or unsubstituted C₉₋₃₁ alkyl which may be saturated or unsaturated.

Sphingolipids of formulas (1), (11), or (12) carry an acyl group deriving from a carboxylic acid, preferably from a fatty acid.

The acyl group carried by the sphingolipids of formulas (1), (11), or (12) can be represented by the acyl group of formula (13):

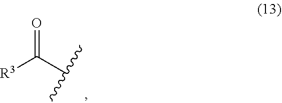

wherein
R³ is selected from a substituted or unsubstituted aryl, a heteroalkyl, a substituted or unsubstituted C₁₋₃₁ alkyl which may be saturated or unsaturated, preferably a substituted or unsubstituted C₉₋₃₁ alkyl which may be saturated or unsaturated.

The person skilled in the art will understand that the acyl group of formula (13) is also carried by the ester of formula (3), (9), or (10).

In some embodiments, R³ of the acyl group of formula (13) is a substituted or unsubstituted C₉₋₃₁ alkyl of formula (5), or (6):

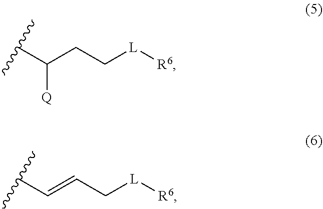

wherein
Q is selected from H, —OH, Cl, Br, I, or F, preferably selected from H, or —OH
L is a linear C₆₋₂₈ alkylene, which may be saturated or contain one or more double and or triple bonds,
R⁶ is H, or —OR⁷, wherein R⁷ is hydrogen or a linear C₂₋₃₀ acyl which may be saturated or contain one or more double bonds.

In some preferred embodiments, R³ of the acyl group of formula (13) is a substituted or unsubstituted C₉₋₃₁ alkyl of formula (5).

Accordingly, in some preferred embodiments, the acyl group of formula (13) is an acyl group of formula (14):

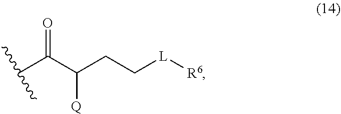

wherein
Q is selected from H, —OH, Cl, Br, I, or F, preferably selected from H, or —OH
L is a linear C₆₋₂₈ alkylene, which may be saturated or contain one or more double and or triple bonds,
R⁶ is H, or —OR⁷, wherein R⁷ is hydrogen or a linear C₂₋₃₀ acyl which may be saturated or contain one or more double bonds.

In some embodiments, R³ of the acyl group of formula (13) is a substituted or unsubstituted C₉₋₃₁ alkyl of formula (6).

Accordingly, in some embodiments, the acyl group of formula (13) is an acyl group of formula (15):

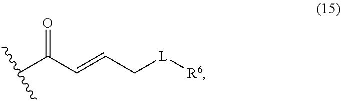

wherein
L is a linear C₆₋₂₈ alkylene, which may be saturated or contain one or more double and or triple bonds,
R⁶ is H, or —OR⁷, wherein R⁷ is hydrogen or a linear C₂₋₃₀ acyl which may be saturated or contain one or more double bonds.

In some embodiments the acyl group of formula (13) or (14) is an acyl group deriving from a C₁₈-C₂₆ non-hydroxy fatty [N].

In some embodiments, the acyl group of formula (13) or (14) is an acyl group deriving from stearic acid [N (18:0)].

In some embodiments, the acyl group of formula (13) or (14) is an acyl group deriving from eicosanoic acid [N (20:0)].

In some embodiments, the acyl group of formula (13) or (14) is an acyl group deriving from behenic acid [N (22:0)].

In some embodiments, the acyl group of formula (13) or (14) is an acyl group deriving from lignoceric acid [N (24:0)].

In some embodiments, the acyl group of formula (13) or (14) is an acyl group deriving from hexacosanoic acid [N (26:0)].

In some embodiments, for the acyl group of formula (14) and for the sphingolipid of formula (16) or (17) Q is-OH, L is a straight-chain saturated unsubstituted C₁₄-C₂₈ alkylene, and R⁸ is H.

In some embodiments the acyl group of formula (13) or (14) is an acyl group deriving from a C₁₈-C₂₆ α-hydroxy fatty acid [A].

In some embodiments, the acyl group of formula (13) or (14) is an acyl group deriving from α-hydroxystearic acid [A (18:0)].

In some embodiments, the acyl group of formula (13) or (14) is an acyl group deriving from α-hydroxyeicosanoic acid [A (20:0)].

In some embodiments, the acyl group of formula (13) or (14) is an acyl group deriving from α-hydroxybehenic acid [A (22:0)].

In some embodiments, the acyl group of formula (13) or (14) is an acyl group deriving from α-hydroxylignoceric acid [A (24:0)].

In some embodiments, the acyl group of formula (13) or (14) is an acyl group deriving from α-hydroxyhexacosanoic acid [A (26:0)].

In some embodiments, for the acyl group of formula (14) and for the sphingolipid of formula (16) or (17) Q is H, L is a straight-chain saturated unsubstituted C₁₄-C₂₈ alkylene, and R⁸ is —OR⁹ wherein R⁹ is a saturated or unsaturated C₁₈ acyl.

In some embodiments, the acyl group carried by the sphingolipid of the present invention is an acyl group of formula (14), wherein the acyl group pf formula (14) is selected from the group consisting of acyl groups of formula (16)-(24):

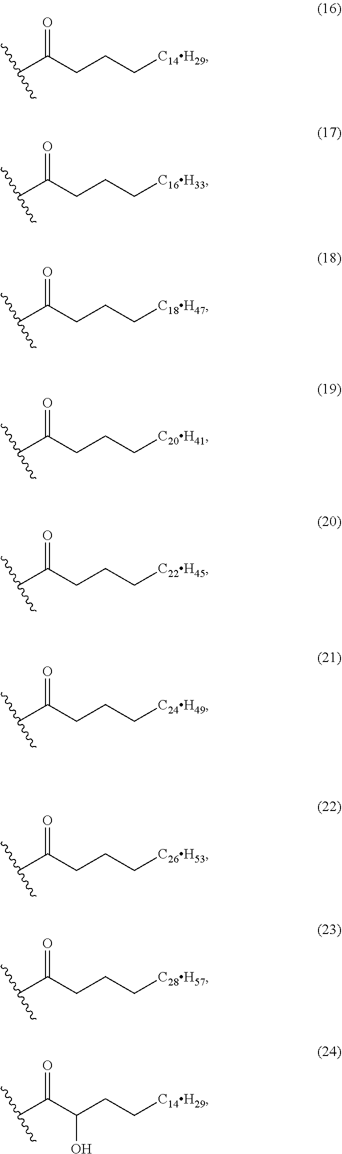

In some preferred embodiments, W of W of the sphingolipid according to the present invention is hydrogen. Accordingly, in some preferred embodiments for the sphingolipid according to the present invention is a ceramide.

Ceramides denote, in the context of the present invention, naturally occurring ceramides, analogues thereof or derivatives thereof. Preferred ceramides are those naturally occurring in humans. Naturally occurring human ceramides (CERs) include, but are not limited to, CER[NS], CER[AS], CER[EOS], CER[NH], CER[AH], or CER[EOH], CER[NP], CER[AP], or CER[EOP], CER[NDS], CER[ADS], or CER[EODS], wherein letters in brackets refer to the shorthand nomenclature developed by Motta et al., Biochim Biophys Acta., 1993, 1182:147-151 and expanded by Rabionet et al., Biochim Biophys Acta, 2014, 1841:422-434, and by Masukawa et al., Journal of Lipid Research, 2008, 49, 1466-1476. The letters N, A, and EO represent non-hydroxy fatty acids (N), alpha-hydroxy fatty acids (A), and omega-linoleoyloxy fatty acids (EO), respectively, wherein the number of fatty acid carbons and unsaturations may be expressed in parentheses following the letters of N, A, E, and O. The letters, S, H, P, and DS represent D-erythro-sphingosine(S), 6-hydroxy-D-erythro-sphingosine (H), D-ribo-phytosphingosine (P), D-erythro-dihydrosphingosine (DS), respectively, wherein the number of sphingoid carbons may be expressed in parenthesis following the letters S, H, P, and DS. Ceramides, CER[NDS], CER[ADS], or CER[EODS], may also be referred to as CER[NG], CER[AG], or CER[EOG], respectively, wherein the letter G represents the INCI name for D-erythro-dihydrosphingosine.

In some embodiments, the sphingolipid of formula (1) or (11) is a ceramide selected from CER[NS], CER[AS], CER[EOS].

In some preferred embodiments, the sphingolipid of formula (1) or (11) is ceramide CER[N(18:0) S(18)].

In some embodiments, the sphingolipid of formula (1) or (11) is ceramide CER[N(20:0) S(18)].

In some embodiments, the sphingolipid of formula (1) or (11) is ceramide CER[N(22:0) S(18)].

In some embodiments, the sphingolipid of formula (1) or (11) is ceramide CER[N(24:0) S(18)].

In some embodiments, the sphingolipid of formula (1) or (11) is ceramide CER[N(26:0) S(18)].

In some preferred embodiments, the sphingolipid of formula (1) or (11) is ceramide CER[A(18:0) S(18)].

In some embodiments, the sphingolipid of formula (1) or (11) is ceramide CER[A(20:0) S(18)].

In some embodiments, the sphingolipid of formula (1) or (11) is ceramide CER[A(22:0) S(18)].

In some embodiments, the sphingolipid of formula (1) or (11) is ceramide CER[A(24:0) S(18)].

In some embodiments, the sphingolipid of formula (1) or (11) is ceramide CER[A(26:0) S(18)].

In some embodiments, the sphingolipid of formula (1) or (12) is a ceramide selected from CER[NH], CER[AH], CER[EOH].

In some preferred embodiments, the sphingolipid of formula (1) or (12) is ceramide CER[N (18:0) H(18)].

In some embodiments, the sphingolipid of formula (1) or (12) is ceramide CER[N(20:0) H(18)].

In some embodiments, the sphingolipid of formula (1) or (12) is ceramide CER[N(22:0) H(18)].

In some embodiments, the sphingolipid of formula (1) or (12) is ceramide CER[N(24:0) H(18)].

In some embodiments, the sphingolipid of formula (1) or (12) is ceramide CER[N(26:0) H(18)].

In some preferred embodiments, the sphingolipid of formula (1) or (12) is ceramide CER[A(18:0) H(18)].

In some embodiments, the sphingolipid of formula (1) or (12) is ceramide CER[A(20:0) H(18)].

In some embodiments, the sphingolipid of formula (1) or (12) is ceramide CER[A(22:0) H(18)].

In some embodiments, the sphingolipid of formula (1) or (12) is ceramide CER[A(24:0) H(18)].

In some embodiments, the sphingolipid of formula (1) or (12) is ceramide CER[A(26:0) H(18)].

In some embodiments, W of the sphingolipid of the present invention and W of the lysosphingolipid of the present invention is a glycosyl moiety, and wherein the glycosyl moiety is selected from Glc1-, Gal1-, Galβ1-4Glc1-.

In some embodiments, W of the sphingolipid of the present invention, and W of the lysosphingolipid of the present invention is a glycosyl moiety, and wherein the glycosyl moiety is the oligosaccharide portion of a ganglioside selected from GM1a, GM1b, GD1a, GD1b, GD3, GT1b, GT3, GQ1b, GM3, GM4.

In the context of the present invention the oligosaccharide portion of GM1a, GM1b, GD1a, GD1b, GD3, GT1b, GT3, GQ1b, GM3 and GM4 may be represented by the following formulas:

Galβ1-3GalNAcβ1-4(Neu5Acα2-3)Galβ1-4Glc1-,
Neu5Acα2-3Galβ1-3GalNAcβ1-4Galβ1-4Glc1-,
Neu5Acα2-3Galβ1-3GalNAcβ1-4(Neu5Acα2-3)Galβ1-4Glc1-,
Galβ1-3GalNAcβ1-4(Neu5Acα2-8Neu5Acα2-3)Galβ1-4Glc1-,
Neu5Acα2-8Neu5Acα2-3Galβ1-4Glc1-,
Neu5Acα2-3Galβ1-3GalNAcβ1-4(Neu5Acα2-8Neu5Acα2-3)Galβ1-
4Glc1-,
Neu5Acα2-8Neu5Acα2-8Neu5Acα2-3Galβ1-4Glc1-,
Neu5Acα2-8Neu5Acα2-3Galβ1-3GalNAcβ1-4(Neu5Acα2-8Neu5Acα2-
3)Galβ1-4Glc1-,
Neu5Acα2-3Galβ1-4Glc1-
Neu5Acα2-3Gal1-,

respectively.

The term, “oligosaccharide portion of a ganglioside” as used herein is defined to encompass glycosyl moieties deriving from gangliosides, wherein the anomeric carbon at the reducing end of the oligosaccharide portion of the ganglioside is engaged in a glycosidic bond with another chemical entity, the glycosidic bond may be an alpha or a beta glycosidic bond, preferably a beta glycosidic bond. In the context of the present invention the terms oligosaccharide portion and glycosyl moiety may be used interchangeably.

In some embodiments, W of the sphingolipid of the present invention, and W of the lysosphingolipid of the present invention is a glycosyl moiety, wherein the glycosyl moiety is that of a human milk oligosaccharide, and wherein the human milk oligosaccharide is preferably selected from LNT, LNnT, LNH, LNnH, 2′FL, 3FL, DFL, LNFP-I, LNFP-II, LNFP-III, LNFP-V, LNDFH-I, 3′SL, 6′SL, FSL, LSTa, LSTb, LSTc, and DSLNT.

In the context of the present invention the glycosyl moieties of LNT, LNnT, LNH, LNnH, 2′FL, 3FL, DFL, LNFP-I, LNFP-II, LNFP-III, LNFP-V, LNDFH-I, 3′SL, 6′SL, FSL, LSTa, LSTb, LSTc, and DSLNT may be represented by the following formulas:

Galβ1-3GlcNAcβ1-3Galβ1-4Glc1-,
Galβ1-4GlcNAcβ1-3Galβ1-4Glc1-,
Galβ1-3GlcNAcβ1-3(Galβ1-4GlcNAcβ1-6)Galβ1-4Glc1-,
Galβ1-4GlcNAcβ1-3Galβ1-4GlucNAcβ1-3Galβ1-4Glc1-,
Fucα1-2Galβ1-4Glc1-,
Galβ1-4(Fucα1-3)Glc1-,
Fucα1-2Galβ1-4(Fucα1-3)Glc1-,
Fucα1-2Galβ1-3GlcNAcβ1-3Galβ1-4Glc1-,
Galβ1-3(Fucα1-4)GlcNAcβ1-3Galβ1-4Glc1-,
Galβ1-3(Fucα1-3)GlcNAcβ1-3Galβ1-4Glc1-,
Galβ1-3GlcNAcβ1-3Galβ1-4(Fucα1-3)Glc1-,
Fucα1-2Galβ1-3(Fucα1-4)GlcNacβ1-3Galβ1-4Glc1-,
Neu5Acα2-3Galβ1-4Glc1-,
Neu5Acα2-6Galβ1-4Glc1-,
Neu5Acα2-3Galβ1-4(Fucα1,3)Glc1-,
Neu5Acα2-3Galβ1-3GlcNAcβ1-3Galβ1-4Glc1-,
Galβ1-3(Neu5Acα2-6)GlcNAcβ1-3Galβ1-4Glc1-,
Neu5Acα2-6Galβ1-3GlcNAcβ1-3Galβ1-4Glc1-,
Neu5Acα2-3Galβ1-3(Neu5Acα2-6)GlcNAcβ1-3Galβ1-4Glc1-,

respectively.

In some embodiments, W of the sphingolipid of the present invention, and W of the lysosphingolipid of the present invention is a glycosyl moiety, and wherein the glycosyl moiety is the oligosaccharide portion of a glycosphingolipid selected from the gala series [SP0509], the neogala series, the globo series [SP0502], the isoglobo series [SP0506], the lacto series [SP0504], the neolacto series [SP0505], the arthro series [SP0508], the muco series, the schisto series, the spirometo series, or protected analogs thereof.

Wherein the bracketed alphanumeric string indicates a link contained on the website https://www.lipidmaps.org/, wherein detailed chemical formulas and structural information for the corresponding compounds can be found.

The sphingolipid produced by the method of the present invention may be isolated from the reaction mixture. The isolation may be performed by standard methods known to the skilled person. A preferred method for the isolation of the sphingolipid according to the present invention is via precipitation from the reaction mixture. Precipitation may be achieved for example via partial removal of the reaction solvent by evaporation, i.e. concentrating the reaction mixture, or via the addition of another solvent to the reaction mixture, or via changes of temperature or pressure, or via addition of other solutes, or combinations of these.

In some embodiments, the sphingolipid according to the present invention is isolated via precipitation from the reaction mixture, wherein the precipitation is achieved via cooling the reaction mixture to a temperature from about 0° C. to about 25° C., preferably from about 0° C. to about 10° C.

The sphingolipids according to the present invention may be isolated in different polymorphic forms. Polymorphic forms as referred to herein can include crystalline and amorphous forms as well as solvate and hydrate forms, which can be further characterized as follows:
i. Crystalline forms have different arrangements and/or conformations of the molecules in the crystal lattice.
ii. Amorphous forms consist of disordered arrangements of molecules that do not possess a distinguishable crystal lattice.
iii. Solvates are crystal forms containing either stoichiometric or non-stoichiometric amounts of a solvent. If the incorporated solvent is water, the solvate is commonly known as a hydrate.

### EXAMPLES

Working examples below describe non-limiting embodiments of the invention and are given only to illustrate the invention.

### General Methods and Material:

¹H NMR and ¹³C NMR was recorded with a Bruker Avance II (400 MHz) spectrometer. ¹H and ¹³C chemical shifts are given in ppm (δ) relative to tetramethylsilane (δ=0.00), CDCl₃ (δ=7.26), CD₃OD (δ=3.31), DMSO-d₅ (δ=2.50) as internal standard. LCMS analysis was performed with SCIEX Triple Quad™ 4500 LC-MS/MS System.

### Example 1. General Procedure for the N-Acylation of Sphingoid Bases

A sphingoid base, or a salt thereof was dissolved in 5-10 volumes of a short-chain aliphatic alcohol, most preferably methanol. The base (0.1-1.7 eq), typically sodium or potassium methoxide, was added and the mixture was stirred for 30-60 minutes at room temperature. An ester of a fatty acid, preferably a fatty acid methyl ester, was added and the reaction mixture was stirred at a temperature between 50° C. and 60° C. until the reaction was completed. The reaction mixture was cooled to a temperature between 25° C. and 18° C. and stirred for 30-120 minutes, and then further cooled to a temperature between 10° C. to 0° C. and stirred for 4-8 hours. A solid suspension formed, this was filtered and washed twice with a short-chain aliphatic alcohol, most preferably methanol, ethanol, propanol, or isopropanol. Typical yield and purity range from 75-98%.

### 1.1 Synthesis of N-stearoyl-D-erythro-sphingosine (25), CER [N(18:0) S(18)]

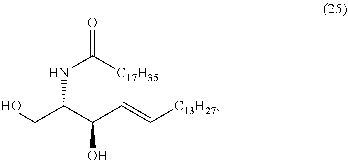

N-stearoyl-D-erythro-sphingosine (25) was synthesized from D-erythro-sphingosine and stearic acid methyl ester following the general procedure of Example 1.

¹H-NMR (500 MHz, CDCl₃) δ (ppm)=6.26 (d, J=7.5 Hz, 1H), 5.82-5.75 (m, 1H), 5.53 (ddt, J=15.3, 6.4, 1.4 Hz, 1H), 4.34-4.30 (m, 1H), 3.95 (dd, J=11.3, 3.8 Hz, 1H), 3.91 (dq, J=7.6, 3.8 Hz, 1H), 3.71 (dd, J=11.3, 3.4 Hz, 1H), 2.35 (s, 2H), 2.26-2.21 (m, 2H), 2.06 (q, J=7.3 Hz, 2H), 1.64 (p, J=7.6 Hz, 2H), 1.26 (s, 53H), 0.91-0.86 (m, 6H)

¹³C-NMR (126 MHz, CDCl₃) δ (ppm)=173.94, 134.32, 128.78, 74.66, 62.50, 54.51, 36.83, 32.27, 31.91, 29.69, 29.68, 29.65, 29.63, 29.61, 29.50, 29.48, 29.36, 29.35, 29.28, 29.22, 29.11, 25.75, 22.68, 14.10.

ESI-MS: 588.55 [M+Na]⁺, 566.61 [M+H]⁺, 548.21 [(M+H)-3H₂O]⁺, 530.58 [(M+H)-2H₂O]⁺, 518.62 [(M+H)-3H₂O]⁺

### 1.2 Synthesis of N-stearoyl 6-hydroxy-D-erythro-sphingosine (26), CER [N(18:0) H(18)]

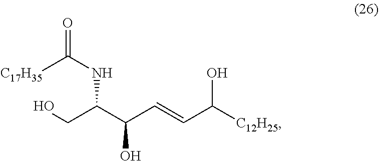

N-stearoyl 6-hydroxy-D-erythro-sphingosine (26) was synthesized from 6-hydroxy-D-erythro-sphingosine and stearic acid methyl ester following the general procedure of Example 1.

ESI-MS: 564.54 [M−H₂O]⁺. 604.45 [M+Na]⁺. 582.50 [M+H]⁺

### 1.3 Synthesis of N-stearoyl-D-erythro-dihydrosphingosine (27), CER[N(18:0) DS(18)]

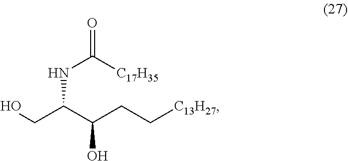

N-stearoyl-D-erythro-dihydrosphingosine (27), was synthesized from D-erythro-dihydrosphingosine and stearic acid methyl ester following the general procedure of Example 1.

¹H-NMR (500 MHz, CD₃OD) δ (ppm)=5.74-5.67 (m, 1H), 5.49-5.43 (m, 1H), 4.36 (d, J=7.8 Hz, 1H), 4.30 (d, J=7.8 Hz, 1H), 4.18 (dd, J=10.1, 4.3 Hz, 1H), 4.11 (t, J=7.2 Hz, 1H), 4.02-3.97 (m, 1H), 3.89-3.81 (m, 3H), 3.75 (dd, J=11.6, 4.6 Hz, 1H), 3.61-3.56 (m, 4H), 3.50 (dd, J=9.6, 3.4 Hz, 1H), 3.44-3.38 (m, 1H), 3.34 (p, J=1.6 Hz, 2H), 3.33-3.30 (m, 1H), 2.21-2.15 (m, 2H), 2.06-1.99 (m, 2H), 1.59 (t, J=7.1 Hz, 2H), 1.40-1.34 (m, 2H), 1.28 (s, 46H), 0.91-0.86 (m, 6H).

¹³C-NMR (126 MHz, CD₃OD) δ (ppm)=211.86, 175.28, 134.75, 130.11, 104.47, 103.72, 80.44, 78.38, 78.12, 77.86, 76.29, 75.73, 75.58, 74.23, 74.05, 72.62, 71.88, 69.65, 69.27, 62.04, 61.52, 54.06, 36.99, 32.88, 32.44, 30.22, 30.20, 30.19, 30.17, 30.16, 30.14, 30.09, 30.08, 29.99, 29.92, 29.85, 29.84, 29.82, 26.49, 23.14, 14.23.

ESI-MS: 568.71 [M+H]⁺, 590.67 [M+Na]⁺.

1. Method for the production of a sphingolipid of formula (1): 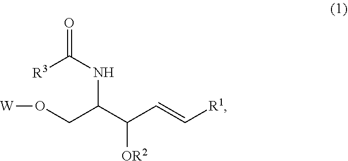
wherein
W is H or a glycosyl moiety,
R¹ is H, aryl, or a C₁₋₅₀ alkyl, preferably a C₁₋₁₅ alkyl, more preferably a C₁₀₋₁₅ alkyl, which may be saturated or contain one or more double and/or triple bonds, and/or which may contain one or more functional groups, the functional group being preferably selected from the group consisting of a hydroxyl group, an alkoxy group, an acyloxy group, an acylamido group, a thiol, a thioether or a phosphorus-containing functional group,
R² is H, a substituted or unsubstituted C₁₋₆ alkyl, or a substituted or unsubstituted C₁₋₆ acyl,
R³ is selected from a substituted or unsubstituted aryl, a heteroalkyl, a substituted or unsubstituted C₁₋₃₁ alkyl which may be saturated or unsaturated, preferably a substituted or unsubstituted C₉₋₃₁ alkyl which may be saturated or unsaturated;
from a lysosphingolipid of formula (2), or a salt thereof: 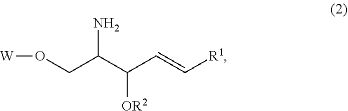
wherein
W, R¹, and R² are as defined as for the sphingolipid of formula (1), and
the method comprising reacting the lysosphingolipid salt of formula (2), or the salt thereof with an ester of formula (3): 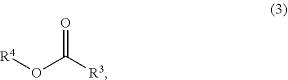
wherein
R³ is as defined as for the sphingolipid of formula (1), and
R⁴ is a C₁-C₄ alkyl, preferably selected from methyl, ethyl, propyl, isopropyl, butyl, or isobutyl, more preferably selected from methyl, or ethyl:
in the presence of a base, in an alcohol solvent.

2. The method according to claim 1, wherein the base is an alkoxide of formula (4): 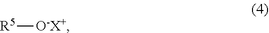
wherein
R⁵ is a C₁-C₄ alkyl, preferably selected from methyl, ethyl, propyl, isopropyl, butyl, or isobutyl, more preferably selected from methyl, or ethyl;
X⁺ is a cation selected from Na⁺, K⁺, Li⁺ or NH₄⁺, preferably Na⁺.

3. The method according to claim 1, wherein about 0.1 to about 1.7 molar equivalents of the base are used, based on the amount of the lysosphingolipid of formula (2).

4. The method according to claim 1, wherein the lysosphingolipid of formula (2) is in a salt form, and wherein about 0.5 to about 1.7 molar equivalents of the base are used, based on the amount of the lysosphingolipid of formula (2).

5. The method according to claim 4, wherein the salt is a hydrochloride, a hydrobromide, a sulfate, a hydrosulfate, a phosphate, a polyphosphate, an acetate, a p-toluene sulfate, a methane sulfate, a trifluoromethanesulfate, or a perchlorate salt of the lysosphingolipid of formula (2), preferably a hydrochloride salt of the lysosphingolipid of formula (2).

6. The method according to claim 1, wherein the lysosphingolipid of formula (2) is in a free-base form, and wherein about 0.1 to about 0.5 molar equivalents of the base are used, based on the amount of the lysosphingolipid of formula (2).

7. The method according to claim 1, wherein the alcohol is a C₁-C₇ alcohol, or a mixture of C₁-C₇ alcohols.

8. The method according to claim 7, wherein the alcohol is C₁-C₃ alcohol, preferably methanol.

9. The method according to claim 1, wherein R² is hydrogen.

10. The method according to claim 1, wherein W is hydrogen, or W is a glycosyl moiety, wherein the glycosyl moiety is preferably selected from Glc1-, Gal1-, Galβ1-4Glc1-.

11. (canceled)

12. The method according to claim 1, wherein R³ is a substituted or unsubstituted C₉₋₃₁ alkyl of formula (5), or (6): 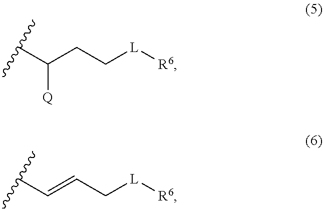
wherein
Q is selected from H, —OH, Cl, Br, I, or F, preferably selected from H, or —OH
L is a linear C₆₋₂₈ alkylene, which may be saturated or contain one or more double and or triple bonds,
R⁶ is H, or —OR⁷, wherein R⁷ is hydrogen or a linear C₂₋₃₀ acyl which may be saturated or contain one or more double bonds.

13. The method according to claim 1, wherein the lysosphingolipid of formula (2) is selected from the group consisting of lysosphingolipids of formula (7) and (8), or salts thereof: 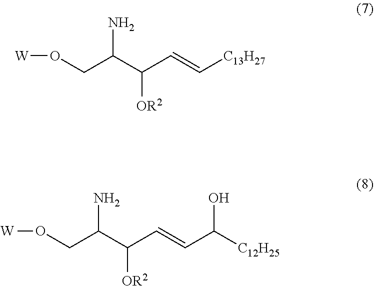
wherein W and R² are as defined as for the lysosphingolipid of formula (2).

14. The method according to claim 1, wherein the lysosphingolipid of formula (2) is selected from the group consisting of is d-erythro-sphingosine, and 6-hydroxy-d-erythro-sphingosine, or salts thereof.

15. The method according to claim 1, wherein the lysosphingolipid of formula (2) is d-erythro-sphingosine, or a salt thereof.

16. The method according to claim 1, wherein the lysosphingolipid of formula (2) is 6-hydroxy-d-erythro-sphingosine, or a salt thereof.

17. The method according to claim 1, wherein the ester of formula (3) is a fatty acid methyl ester selected from the group consisting of stearic acid methyl ester, eicosanoic acid methyl ester, behenic acid methyl ester, lignoceric acid methyl ester, hexacosanoic acid methyl ester, α-hydroxystearic acid methyl ester, α-hydroxyeicosanoic acid methyl ester, α-hydroxybehenic acid methyl ester, α-hydroxylignoceric acid methyl ester, and α-hydroxyhexacosanoic acid methyl ester.

18. The method according to claim 1, wherein the sphingolipid of formula (1) is a ceramide selected from the group consisting of CER[N(18:0) S(18)], CER[N(20:0) S(18)], CER[N(22:0) S(18)], CER[N(24:0) S(18)], CER[N(26:0) S(18)], CER[N(18:0) H(18)], CER[N(20:0) H(18)], CER[N(22:0) H(18)], CER[N(24:0) H(18)], and CER[N(26:0) H(18)].

19. The method according to claim 18, wherein the sphingolipid of formula (1) is a ceramide selected from the group consisting of CER[N(18:0) S(18)], CER[N(18:0) H(18)].

20. The method according to claim 1, wherein the sphingolipid of formula (1) is a ceramide selected from the group consisting of CER[A(18:0) S(18)], CER[A(20:0) S(18)], CER[A(22:0) S(18)], CER[A(24:0) S(18)], CER[A(26:0) S(18)], CER[A(18:0) H(18)], CER[A(20:0) H(18)], CER[A(22:0) H(18)], CER[A(24:0) H(18)], CER[A(26:0) H(18)].

21. The method according to claim 20, wherein the sphingolipid of formula (1) is a ceramide selected from the group consisting of CER[A(18:0) S(18)], CER[A(18:0) H(18)].
